(19) United States
(12) Patent Application Publication
(10) Pub. No.: US 2019/0071680 A1
(43) Pub. Date: Mar. 7, 2019

(54) MICROBIAL PRODUCTION OF NICOTINIC ACID RIBOSIDE

(71) Applicant: DSM IP Assets B.V., Heerlen (NL)

(72) Inventors: Adam G. LAWRENCE, Kaiseraugst (CH); Celine VIAROUGE, Kaiseraugst (CH)

(21) Appl. No.: 16/084,305

(22) PCT Filed: Mar. 16, 2017
(86) PCT No.: PCT/EP2017/056329

§ 371 Date: Sep. 12, 2018

Related U.S. Application Data
(60) Provisional application No. 62/309,238, filed on Mar. 16, 2016.

(51) Int. Cl. C12N 15/52; A23L 33/135; A23L 33/15; C12N 9/06; C12N 9/12; C12N 9/10; C12N 9/82; C12R 1/19; C12R 1/125; C12R 1/15

(52) U.S. Cl. CPC C12N 15/52; C12Y 305/01001; A23L 33/15; C12N 9/0022; C12N 9/1241; C12N 9/1205; C12N 9/1077; C12N 9/82; C12N 9/1085; C12R 1/19; C12R 1/125; C12R 1/15; C12Y 104/03016; C12Y 104/03001; C12Y 204/02019; C12Y 204/02012; C12Y 205/01; C12Y 207/07001; C12Y 207/01173; A23L 33/135

(57) ABSTRACT

The present invention relates to a novel method, expression vectors, and host cells for producing nicotinic acid riboside by regulating the pathways that lead to the production of nicotinic acid riboside.

The present invention relates to a novel method, expression vectors, and host cells for producing nicotinic acid riboside by regulating the pathways that lead to the production of nicotinic acid riboside.

Nicotinic acid riboside (NaR) is a pyridine-nucleoside form of vitamin B3 that functions as a precursor to nicotinamide adenine dinucleotide or NAD+. It is believed that high dose nicotinic acid (NA) can help to elevate high-density lipoprotein cholesterol, and lowers low-density lipoprotein cholesterol and free fatty acids, although its mechanism has not been completely understood. However, high dose NA induces an undesirable flushing response. Like NA and

NR, NaR can be converted to NAD+ in the mammalian cell. However, research into the potential benefits of NaR to human and animal nutrition has been blocked because an efficient and economical method for its production has not been described. Thus, it is desirable to identify new methods for producing nicotinic acid riboside (NaR).

The biosynthesis of NAD+ in bacteria was first elucidated in the 1990s, and was shown to depend on two key enzymatic activities which are not found in eukaryotes: an FAD dependent L-aspartate oxidase (E. coli NadB, EC 1.4.3.16); and a quinolate synthase (E. coli NadA, EC 2.5.1.72). L-aspartate oxidase catalyzes the oxidation of L-aspartate to iminosuccinic acid, utilizing molecular oxygen as an electron acceptor and producing hydrogen peroxide, with the involvement of a loosely bound flavin adenine dinucleotide (FAD) cofactor. The enzyme in Escherichia coli is known to be inhibited by the downstream product NAD+, but feedback resistant mutants have been generated. Quinolate synthase, which contains an iron-sulfur cluster, subsequently carries out the condensation and cyclization of iminosuccinic acid with dihydroxyacetone phosphate yielding quinolate. The combined activity of these two enzymes will produce one mole of quinolate from one mole of aspartate and one mole of dihydroxyacetone phosphate.

Three further enzymatic activities are common to the two canonical de novo pathways of NAD+ synthesis: quinolate phosphoribosyltransferase (E. coli NadC, EC 2.4.2.19); nicotinate mononucleotide adenyltransferase (E. coli NadD, EC 2.7.7.18); and nicotinic acid mononucleotide adenyltransferase, a.k.a. NAD+ synthetase (E. coli NadE, EC 6.3.1.5). Quinolate phosphoribosyltransferase transfers the phosphoribosyl moiety from phosphoribosylpyrophosphate to the quinolate nitrogen and catalyzes the subsequent decarboxylation of the intermediate to produce nicotinic acid mononucleotide (NaMN), pyrophosphate, and carbon dioxide. Nicotinic acid mononucleotide adenyltransferase (NMNAT) uses adenine triphosphate (ATP) to adenylate NaMN, producing nicotinic acid dinucleotide (NaAD) and pyrophosphate. The final step in NAD+ biosynthesis is catalyzed by NAD+ synthetase, which utilizes either ammonia or glutamine as a nitrogen donor to amidate NaAD to NAD+, hydrolyzing one mole of ATP to AMP and pyrophosphate.

In addition to the de novo pathways, there exist multiple characterized pathways for the salvage of NaR, NR, NMN, nicotinamide (Nam) or nicotinic acid (NA). These salvage pathways have been manipulated in Saccharomyces cerevisiae to enable production of NR (U.S. Pat. No. 8,114,626). Simultaneous deletion of the S. cerevisiae genes nrk1, urh1, and pnp1 increased extracellular NR ˜10 fold and additional deletion of nrt1 resulted in a further ˜4 fold increase to 4 uM. These genes respectively encode the activities for NR kinase (E. coli NadR), purine nucleoside phosphorylase (both urh1 and pnp1, E. coli DeoD) and NR transport (E. coli pnuC).

Expression of nad genes is typically co-regulated in bacteria by a transcriptional repressor. In E. coli, transcription of nadA, nadB, and pncB is repressed by the NadR protein, which also has catalytic activities that contribute to salvage pathways. NadR blocks transcription by binding to a conserved motif in the presence of NAD+. In Bacillus subtilis, a different protein named YrxA performs a similar role, by blocking the transcription of two divergently transcribed operons, nadB-nadA-nadC and nifS-yrxA, in the presence of NA (Rossolillo, 2005, J. Bacteriol., 187(20), 7155-7160).

Thus, there is an ongoing need to find more effective ways to increase the production of nicotinic acid.

The inventors have now surprisingly found a novel method for significantly increasing the production of nicotinic acid riboside and created host cells and expression vectors useful in such methods.

The present invention is directed to a genetically modified bacterium capable of producing nicotinic acid riboside (NaR), wherein the bacterium comprises at least one modification selected from a group consisting of: a) blocking or reducing the activity of a protein which functions to repress NAD+ biosynthesis by repressing transcription of nadA, nadB, nadC genes or combinations thereof; b) adding or increasing the transcription of a gene which encodes L-aspartate oxidase, quinolate synthase, quinolate phoshoribosyltransferase, or combinations thereof; and c) blocking or reducing the activity of a protein which functions as a nicotinic acid mononucleotide adenyltransferase; wherein the bacterium with said at least one modification produces an increased amount of NaR than the bacterium without any of said modifications.

Optionally, the bacterium may further comprise at least one modification selected from a group consisting of: d) blocking or reducing the activity of a protein which functions as a nucleoside phosphorylase; e) blocking or reducing the activity of a protein which functions as a nicotinic acid riboside kinase; f) blocking or reducing the activity of a protein which functions as a nicotinic acid riboside transport protein; g) blocking or reducing the activity of a protein which functions as a nicotinic acid phosphoribosyl transferase; h) adding or increasing the activity of a protein which functions as a nicotinamide mononucleotide amidohydrolase; and i) adding or increasing the activity of a protein which functions as a nicotinic acid mononucleotide hydrolase.

In some embodiments, the negative regulator of NAD+ biosynthesis is a polypeptide comprising an amino acid sequence of any one of SEQ ID NO: 1, 2 or 3 or a variant of said polypeptide, wherein said polypeptide has an activity for repressing NAD+ biosynthesis

In some embodiments, the quinolate synthase is a polypeptide comprising an amino acid sequence of any one of SEQ ID NO: 23, 24, or 25 or a variant of said polypeptide, wherein said polypeptide has an activity of converting iminosuccinic acid and dihydroxyacetone phosphate to quinolate and phosphate.

In some embodiments, the L-aspartate oxidase is a polypeptide comprising an amino acid sequence of any one of SEQ ID NO: 26 or 27 or a variant of said polypeptide, wherein said polypeptide has an activity of converting aspartic acid to iminosuccinic acid in an FAD dependent reaction.

In some embodiments, the quinolate phosphoribosyltransferase is a polypeptide comprising an amino acid sequence of any one of SEQ ID NO: 28, 29 or 30 or a variant of said polypeptide, wherein said polypeptide has an activity of converting quinolate and phosphoribosylpyrophosphate to nicotinamide mononucleotide and carbon dioxide.

In some embodiments, the nicotinic acid mononucleotide adenyltransferase protein is a polypeptide comprising an amino acid sequence of any one of SEQ ID NO: 4, 5, or 6 or a variant of said polypeptide, wherein said polypeptide has a nictonic acid mononucleotide adenyltransferase activity for converting nicotinic acid mononucleotide to nicotinic acid adenine dinucleotide.

In some embodiments, nicotinic acid riboside phosphorylase is a polypeptide comprising an amino acid sequence of any one of SEQ ID NO: 7, 8, 18, or 19 or a variant of said polypeptide, wherein said polypeptide has a nucleoside cleavage activity for converting nicotinic acid riboside to nicotinic acid and ribose phosphate.

In some embodiments, the nicotinic acid riboside transporter is a polypeptide comprising an amino acid sequence of any one of SEQ ID NOs: 9, 10, or 11 or a variant of said polypeptide, wherein said polypeptide has a nicotinic acid riboside transport activity for importing nicotinic acid riboside.

In some embodiments, the nicotinic acid mononucleotide hydrolase is a polypeptide comprising an amino acid sequence of any one of SEQ ID NOs: 12, 13, or 14 or a variant of said polypeptide, wherein said polypeptide has a nicotinic acid mononucleotide hydrolase activity for converting nicotinic acid mononucleotide to nicotinic acid riboside.

In some embodiments, the nicotinate phosphoribosyl transferase is a polypeptide comprising an amino acid sequence of any one of SEQ ID NOs: 15, 16, or 17 or a variant of said polypeptide, wherein said polypeptide has a nicotinic acid phosphoribosyl transferase activity for converting nicotinic acid, 5-phospho-ribose 1-diphosphate, and adenosine triphosphate to nicotinic acid mononucleotide, adenosine diphosphate, diphosphate and phosphate.

In some embodiments, the nicotinic acid riboside kinase is a polypeptide comprising an amino acid sequence, SEQ ID NO 1, wherein said polypeptide has nicotinic acid riboside kinase activity for converting nicotinic acid riboside to nicotinic acid mononucleotide.

In some embodiments, the nicotinamide mononucleotide amidohydrolase is a polypeptide comprising an amino acid sequence of any one of SEQ ID NOs:20, 21, or 22 or a variant of said polypeptide, wherein said polypeptide has a nicotinamide mononucleotide amidohydrolase activity for converting nicotinamide mononucleotide to nicotinic acid mononucleotide.

In some embodiments, the genetically modified bacterium may be an E. coli, B. subtilis, C. glutamicum, A. baylyi or a R. eutropha.

The present invention is also directed to a method for producing NaR, comprising: culturing a bacterium cell under conditions effective to produce NaR and recovering NaR from the medium and thereby producing NaR, wherein the host microorganism comprises at least one modification selected from the group consisting of: a) blocking or reducing the activity of a protein which functions to repress NAD+ biosynthesis by repressing transcription of nadA, nadB, nadC genes or combinations thereof; b) adding or increasing the transcription of a gene which encodes L-aspartate oxidase, quinolate synthase, quinolate phosphoribosyltransferase, or combinations thereof; and c) blocking or reducing the activity of a protein which functions as a nicotinic acid mononucleotide adenyltransferase. Optionally, the bacterium may further comprise at least one modification selected from a group consisting of: d) blocking or reducing the activity of a protein which functions as a nicotinic acid riboside phosphorylase; e) blocking or reducing the activity of a protein which functions as a nicotinic acid riboside kinase; f) blocking or reducing the activity of a protein which functions as a nicotinic acid riboside transport protein; g) blocking or reducing the activity of a protein which functions as a nicotinate phosphoribosyl transferase; h) adding or increasing the activity of a protein which functions as a nicotinamide mononucleotide amidohydrolase; and i) adding or increasing the activity of a protein which functions as a nicotinic acid mononucleotide hydrolase.

The present invention is also directed to nicotinic acid riboside compounds obtained from any of the above mentioned genetically modified bacterium.

The present invention is also directed to a composition comprising the nicotinic acid riboside compounds obtained from the above-mentioned genetically modified bacterium.

The present invention is also directed to a food product or feed comprising the nicotinic acid riboside compounds obtained from the above-mentioned genetically modified bacterium.

Unless otherwise defined herein, scientific and technical terms used herein will have the meanings that are commonly understood by one of ordinary skill in the art.

The term “quinolate synthase” indicates an enzyme capable of converting iminosuccinic acid and dihydroxyacetone phosphate to quinolate and phosphate, see FIG. 1. The quinolate synthase used in this invention can be from various organisms, such as E. coli, B. subtilis, C. glutamicum, etc. Examples of quinolate synthase proteins include polypeptides having amino acid sequence SEQ ID NO:23, 24, or 25. Genes encoding the quinolate synthesis activity are provided under, for example, accession nos. ACX40525 (E. coli), NP_390663 (B. subtilis), and CAF19774 (C. glutamicum). The quinolate synthase as defined includes functional variants of the above mentioned quinolate synthases.

The term “L-aspartate oxidase” indicates an enzyme capable of converting aspartic acid to iminosuccinic acid in an FAD dependent reaction. The L-aspartate oxidase used in this invention can be from various organisms, such as E. coli, B. subtilis, C. glutamicum, etc. Examples of nucleoside hydrolase proteins include polypeptides having amino acid sequence SEQ ID NO:26 or 27. Genes encoding the L-aspartate oxidase activity are provided under, for example, accession nos. ACX38768 (E. coli) and NP_390665 (B. subtilis). The L-aspartate oxidase as defined includes functional variants of the above mentioned L-aspartate oxidases.

The term “quinolate phosphoribosyl transferase” indicates an enzyme capable of converting quinolate and phosphoribosylpyrophosphate to nicotinamide mononucleotide and carbon dioxide. The quinolate phosphoribosyl transferase used in this invention can be from various organisms, such as E. coli, B. subtilis, C. glutamicum, etc. Examples of nucleoside hydrolase proteins include polypeptides having amino acid sequence SEQ ID NO:28, 29, or 30. Genes encoding the quinolate phosphoribosyl transferase activity are provided under, for example, accession nos. ACX41108 (E. coli), NP_390664 (B. subtilis), and CAF19773 (C. glutamicum). The quinolate phosphoribosyl transferase as defined includes functional variants of the above mentioned quinolate phosphoribosyl transferases.

The term “negative regulator of NAD+ biosynthesis” indicates an enzyme capable of repressing the NAD+ biosynthesis activity by repressing transcription of quinolate synthase (NadA), FAD dependent L-aspartate oxidase (NadB), quinolate phosphoribosyltransferase (NadC), or any combination thereof. The negative regulator of NAD+ biosynthesis protein described in this invention can be from various organisms, such as E. coli, B. subtilis, C. glutamicum, etc. Examples of negative regulator of NAD+ biosynthesis proteins include polypeptides having amino acid sequences SEQ ID NO:1, 2, or 3. Genes encoding the negative regulator of NAD+ biosynthesis activity are provided under, for example, accession nos. NP_418807 (E. coli), P39667 (B. subtilis), and BAF54131 (C. glutamicum). The negative regulator of NAD+ biosynthesis as defined includes functional variants of the above mentioned negative regulators of NAD+ biosynthesis.

The term “nicotinic acid mononucleotide adenyltransferase” indicates an enzyme capable of catalyzing the conversion of nicotinic acid mononucleotide to nicotinic acid adenine dinucleotide. The enzyme is known as NadD in E. coli. The nicotinic acid mononucleotide adenyltransferase protein described in this invention can be from various organisms, such as E. coli, B. subtilis, C. glutamicum, etc. Examples of nicotinic acid mononucleotide adenyltransferase proteins include polypeptides having amino acid sequences SEQ ID NOs: 4, 5, or 6. Genes encoding the nicotinic acid mononucleotide adenyltransferase activity are provided under, for example, accession nos. NP_415172 (E. coli), NP_390442 (B. subtilis), and CAF21017 (C. glutamicum). The nicotinic acid mononucleotide adenyltransferase as defined includes functional variants of the above mentioned nicotinic acid mononucleotide adenyltransferases.

The term “nicotinic acid riboside phosphorylase” indicates an enzyme capable of catalyzing the conversion of nicotinic acid riboside to nicotinic acid and ribose phosphate. The enzyme is known as PncB in E. coli. The nicotinic acid riboside phosphorylase protein described in this invention can be from various organisms, such as E. coli, B. subtilis, C. glutamicum, etc. Examples of nicotinic acid riboside phosphorylase proteins include polypeptides having amino acid sequences SEQ ID NOs: 7, 8, 18 or 19. Genes encoding nicotinic acid riboside phosphorylase activity are provided under, for example, accession nos. NP_418801 (E. coli), NP_389844 (B. subtilis), NP_390230 (B. subtilis), and NP_391819 (B. subtilis). The nicotinic acid riboside phosphorylase as defined includes functional variants of the above mentioned nicotinic acid riboside phosphorylases.

The term “nicotinic acid riboside transporter protein” indicates a protein capable of catalyzing the transport of nicotinic acid riboside for importing nicotinic acid riboside from the periplasm to the cytoplasm. The enzyme is known as PnuC in E. coli. The nicotinic acid riboside transporter protein described in this invention can be from various organisms, such as E. coli, B. subtilis, C. glutamicum, etc. Examples of nicotinic acid riboside transporter proteins include polypeptides having amino acid sequences SEQ ID NO:9, 10, or 11. Genes encoding the NaR transport activity are provided under, for example, accession nos. CAG67923 (A. baylyi), NP_599316 (C. glutamicum), and NP_415272 (E. coli). The nicotinic acid riboside transporter protein as defined includes functional variants of the above mentioned nicotinic acid riboside transporter proteins.

The term “nicotinic acid mononucleotide hydrolase” indicates an enzyme capable of catalyzing the hydrolysis of nicotinic acid mononucleotide to nicotinic acid riboside. The enzyme is known as UshA in E. coli. The nucleoside hydrolase used in this invention can be from various organisms, such as E. coli, B. subtilis, C. glutamicum, etc. Examples of nucleoside hydrolase proteins include polypeptides having amino acid sequence SEQ ID NO:12, 13, or 14. Genes encoding the nucleoside hydrolase activity are provided under, for example, accession nos. NP_415013 (E. coli), NP_388665 (B. subtilis), and CAF18899 (C. glutamicum). The nicotinic acid mononucleotide hydrolase as defined includes functional variants of the above mentioned nicotinic acid mononucleotide hydrolases.

The term “nicotinic acid phosphoribosyl transferase” indicates an enzyme capable of catalyzing the conversion of nicotinic acid, 5-phospho-ribose 1-diphosphate, and adenosine triphosphate to nicotinic acid mononucleotide, adenosine diphosphate, diphosphate and phosphate. The enzyme also catalyzes the reverse reaction. The nicotinic acid phosphoribosyl transferase protein described in this invention can be from various organisms, such as E. coli, B. subtilis, C. glutamicum, etc. Examples of nicotinic acid phosphoribosyl transferase proteins include polypeptides having amino acid sequences SEQ ID NOs: 15, 16 or 17. Genes encoding the nicotinic acid phosphoribosyl transferase activity are provided under, for example, accession nos. NP_415451 (E. coli), NP_391053 (B. subtilis), and CAF21180 (C. glutamicum). The nicotinic acid phosphoribosyl transferase as defined includes functional variants of the above mentioned nicotinic acid phosphoribosyl transferases.

The term “nicotinamide mononucleotide amidohydrolase” indicates an enzyme capable of catalyzing the conversion of nicotinamide mononucleotide to nicotinic acid mononucleotide. The enzyme is known as PncC in E. coli. The nicotinamide mononucleotide amidohydrolase described in this invention can be from various organisms, such as E. coli, B. subtilis, C. glutamicum, etc. Examples of nicotinamide mononucleotide amidohydrolase proteins include polypeptides having amino acid sequences SEQ ID NOs: 20, 21, and 22. Genes encoding the nicotinamide mononucleotide amidohydrolase activity are provided under, for example, accession nos. NP_417180 (E. coli), AAB00568 (B. subtilis), and CAF20304 (C. glutamicum). The nicotinamide mononucleotide amidohydrolase as defined includes functional variants of the above mentioned nicotinamide mononucleotide amidohydrolases.

The term “nicotinic acid riboside kinase” indicates an enzyme capable of catalyzing the conversion of nicotinic acid riboside to nicotinic acid mononucleotide. The nicotinic acid riboside kinase protein described in this invention can be from various organisms, such as E. coli, B. subtilis, C. glutamicum, etc. Examples of a nicotinic acid riboside kinase proteins include polypeptides having the amino acid sequences SEQ ID NO:1. Genes encoding the nicotinic acid riboside kinase activity are provided under, for example, accession no. NP_418807 (E. coli). The nicotinic acid riboside kinase as defined includes functional variants of the above mentioned nicotinic acid riboside kinase.

Sequence Identity: The relatedness between two amino acid sequences or between two nucleotide sequences is described by the parameter “sequence identity”.

For purposes of the present disclosure, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the-nobrief option) is used as the percent identity and is calculated as follows:

(Identical Residues×100)/(Length of Alignment−Total Number of Gaps in Alignment)

For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the-nobrief option) is used as the percent identity and is calculated as follows:

(Identical Deoxyribonucleotides×100)/(Length of Alignment−Total Number of Gaps in Alignment)

Nucleic acid construct: The term “nucleic acid construct” means a nucleic acid molecule, either single or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

Control sequences: The term “control sequences” means all components necessary for the expression of a polynucleotide encoding a polypeptide of the present disclosure. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Host cell: The term “host cell” means any bacterial cell type that is susceptible to transformation, transfection, transduction, and the like with a nucleic acid construct or expression vector comprising a polynucleotide encoding any one of the polypeptide sequences of the present disclosure. The term “host cell” encompasses any progeny of a parent cell that is not identical to the parent cell due to mutations that occur during replication.

The present invention features bacterial strains with genetically engineered features for the production of nicotinic acid riboside.

Production of nicotinamide riboside has been described in yeast by modification of salvage pathways. Surprisingly, combining modifications to NAD+ biosynthetic and salvage pathways in bacteria result in production of nicotinic acid riboside.

Accordingly, in the first embodiment of the invention, it is desirable to introduce one or more genetic modifications resulting in increased rates of production of nicotinic acid mononucleotide within a host cell. The modification may include deletion or reduction in expression of a gene that represses transcription of all or some of the genes of the de novo NAD+ biosynthetic pathway, nadA, nadB, and/or nadC. See FIG. 2. The modification may also or alternatively include increasing the expression of the L-aspartate oxidase gene, the quinolate synthase gene, quinolate phosphoribosylpyrophosphate gene, or combinations thereof, encoded, for example, by nadB (E. coli, B. subtilis), nadA (E. coli, B. subtilis, C. glutamicum), or nadC (E. coli, B. subtilis, C. glutamicum). The modification may also or alternatively include modifications to the nadB gene which render the gene resistant to inhibition by the downstream metabolite NAD+.

In E. coli, B. subtilis, C. glutamicum, and other species of bacteria, NaMN is converted to nicotinic acid adenine dinucleotide (NaAD) by the action of the nicotinic acid mononucleotide adenyltransferase (NMNAT, EC 2.7.7.18). Reduction in activity of the NMNAT causes increased levels of intracellular NaMN which results in increased NaMN export and dephosphorylation to NaR. Thus, in a second embodiment, one or more genetic modifications are introduced to the host cell to decrease NMNAT activity. In certain preferred embodiments, the reduction in NMNAT activity is accomplished through changes to the amino acid sequence of the polypeptide which has NMNAT activity. For example, in certain embodiments, the modification may comprise changes to the threonine encoded at position 10 or the asparagine encoded at position 39 of the B. subtilis nadD gene (SEQ ID NO:6) to another amino acid. In other embodiments the modification may comprise a change of the threonine encoded at position 11 or the asparagine encoded at position 40 of the E. coli nadD gene (SEQ ID NO:7) to another amino acid. In other embodiments, the modification may comprise a change of the threonine encoded at position 25 of the C. glutamicum nadD gene (SEQ ID NO:8) to another amino acid. The modification may also comprise modifications to the region 5′ or 3′ of the open reading frame encoding the NMNAT such that transcription and/or translation of the gene occurs with lower efficiency.

In some embodiments, the negative regulator of NAD+ biosynthesis is a polypeptide comprising an amino acid sequence of either SEQ ID NO: 1, 2 or 3 or a variant of said polypeptide, wherein said polypeptide has an activity for repressing NAD+ biosynthesis.

In some embodiments, the quinolate synthase is a polypeptide comprising an amino acid sequence of either SEQ ID NO: 23, 24, or 25 or a variant of said polypeptide, wherein said polypeptide has an activity of forming quinolate from iminosuccinic acid and dihydroxyacetone phosphate.

In some embodiments, the L-aspartate oxidase is a polypeptide comprising an amino acid sequence of either SEQ ID NO: 26 or 27 or a variant of said polypeptide, wherein said polypeptide has an activity of forming iminosuccinic acid from aspartic acid.

In some embodiments, the quinolate phosphoribosyltransferase is a polypeptide comprising an amino acid sequence of either SEQ ID NO: 28, 29 or 30 or a variant of said polypeptide, wherein said polypeptide has an activity of forming nicotinic acid mononucleotide from quinolate and phosphoribosylpyrophosphate.

In some embodiments, the nicotinic acid mononucleotide adenyltransferase protein is a polypeptide comprising an amino acid sequence of either SEQ ID NO: 4, 5, or 6 or a variant of said polypeptide, wherein said polypeptide has a nictonic acid mononucleotide adenyltransferase activity for converting nicotinic acid mononucleotide to nicotinic acid adenine dinucleotide.

The present invention further embraces a genetically engineered bacterial strain deficient in nicotinic acid riboside import and salvage pathways. See FIG. 4. Disruption of the NaR salvage pathway in bacteria is expected to result in accumulation of extracellular NaR, because such a strain would fail to import NaR into the cytoplasm and would also fail to phosphorylate NaR into NaMN, or to degrade NaR into nicotinic acid (NA) and ribose phosphate, either intra- or extracellularly. Four enzymatic activities are of particular importance for engineering bacterial NaR production. The phosphorylation of intracellular NaR by nicotinic acid riboside kinase recycles this compound back into the NAD+ biosynthetic pathway. Removal of this activity by deletion or reduced expression of the gene encoding the NR kinase activity, such as nadR in E. coli, will increase the intracellular pool of NaR by preventing its conversion to NaMN. The degradation of NaR to NA and ribose phosphate by the nucleoside phosphorylase activity removes product and deletion or decreased expression of the gene encoding this activity, for example deoD in E. coli or pdp in B. subtilis, will increase rates of product formation. Nicotinic acid and 5-phospho-α-D-ribose 1-diphosphate are ligated to nicotinic acid mononucleotide by the nicotinate phosphoribosyl transferase activity. This reaction is reversible under physiological conditions and may serve to reroute NaMN from hydrolysis to NaR into the side product nicotinic acid. Reduced expression or deletion of the gene encoding this activity, for example, pncB in E. coli, yueK in B. subtilis or cg2774 in C. glutamicum will increase NaR formation by prevented this degradative reaction. A transport protein is responsible for import of extracellular NaR and deletion or reduced expression of the gene encoding this activity, for example pnuC in E. coli or nupC in B. subtilis, will produce an increase in rates of production of extracellular NaR.

Accordingly, in a third embodiment of the invention, it is desirable to reduce or block the nicotinamide riboside import and salvage pathways and thus cause the host cell to preserve the nicotinamide riboside that has been produced. In certain embodiments, bacterial strains of this invention possess one or more of the following features: i) a blocked or reduced protein which functions as a nicotinic acid riboside phosphorylase; ii) a blocked or reduced protein which functions as a nicotinic acid riboside kinase; iii) a blocked or reduced protein which functions as a nicotinic acid riboside transport protein; iv) a blocked or reduced protein which functions as a nicotinic acid phosphoribosyl transferase.

The nicotinic acid riboside phosphorylase according to embodiments herein may include, for example and without limitation, a polypeptide comprising an amino acid sequence of either SEQ ID NO: 7, SEQ ID NO: 8, SEQ ID NO:18, or SEQ ID NO: 19 or a variant of said polypeptide, wherein the above polypeptide has the activity of nicotinic acid riboside phosphorylase for converting nicotinic acid riboside to nicotinic acid and ribose phosphate.

The nicotinic acid riboside kinase according to embodiments herein may include, for example and without limitation, a polypeptide comprising an amino acid sequence of SEQ ID NO: 1 or a variant of said polypeptide, wherein the above polypeptide has the activity nicotinic acid kinase for converting nicotinic acid riboside to nicotinic acid mononucleotide.

The nicotinic acid riboside uptake transporter according to embodiments herein may include, for example and without limitation, a polypeptide comprising an amino acid sequence of any one of SEQ ID NOs: 9, 10 or 11 or a variant of said polypeptide, wherein the above polypeptide has nicotinic acid riboside importing activity.

The nicotinic acid mononucleotide phosphoribosyl transferase according to embodiments herein may include, for example and without limitation, a polypeptide comprising an amino acid sequence of either SEQ ID NO: 15, SEQ ID NO:16, or SEQ ID NO:17 or a variant of said polypeptide, wherein the above polypeptide has the activity of nicotinic acid mononucleotide phosphoribosyl transferase for converting nicotinic acid, 5-phospho-ribose 1-diphosphate, and adenosine triphosphate to nicotinic acid mononucleotide, adenosine diphosphate, diphosphate and phosphate in a reversible reaction.

In a fourth embodiment of the invention, it is desirable to increase the expression level of the extracellular nicotinic acid mononucleotide hydrolase gene, encoded, for example, by ushA in E. coli or by yfkN in B. subtilis and thus to cause the host cell to produce excess extracellular NaR from NaMN. See FIG. 3. In one embodiment, the invention is directed to a bacterial strain having an increased activity of the nicotinic acid mononucleotide hydrolase. The nicotinamide mononucleotide hydrolase according to embodiments herein may include, for example and without limitation, a polypeptide comprising an amino acid sequence of SEQ ID NOs: 12, 13 or 14 or a variant of said polypeptide, wherein the above polypeptide has the activity of nicotinic acid mononucleotide hydrolase for converting nicotinic acid mononucleotide to nicotinic acid riboside.

In addition to serving as a cofactor, NAD+ is consumed in a variety of cellular process, for example as substrate for bacterial DNA ligase, with the concomitant release of NMN. In a fifth embodiment of the invention, rapid recycling of NMN to NaMN biases the system towards production of NaR over NR and is accomplished by overexpression of nicotinamide mononucleotide amidohydrolase, encoded, for example by pncC in E. coli (SEQ ID NO: 20), CinA in B. subtilis (SEQ ID NO:21), or cg2153 in C. glutamicum (SEQ ID NO:22), or a variant of said nicotinamide mononucleotide amidohydrolase.

In other embodiments, the bacterial strains described in the above first or second embodiment further comprise the above third embodiment, fourth embodiment, or both.

For example, in one embodiment, the present invention is directed to a genetically modified bacterium capable of producing nicotinic acid riboside, wherein the bacterium comprises the following modifications: i) altered or increased L-aspartate oxidase activity, altered or increased quinolate synthase activity, altered or increased quinolate phosphoribosylpyrophosphate activity or combinations thereof, in a host with an altered negative regulator of NAD+ biosynthesis with a blocked or reduced activity and ii) one or more additional modifications selected from the group consisting of: d) an altered nicotinic acid riboside kinase with a blocked or reduced activity; e) an altered nicotinic acid riboside phosphorylase with a blocked or reduced activity; f) an altered nicotinamide riboside uptake transporter with a blocked or reduced activity; g) an altered nicotinic acid phosphoribosyl transferase with a blocked or reduced activity; h) an altered nicotinamide mononucleotide amidohydrolase with an added or increased activity; and i) an altered nicotinic acid mononucleotide hydrolase with an added or increased activity; wherein the bacterium with said at least one modification produces an increased amount of NaR than the bacterium without any of said modifications.

For example, in one embodiment, the present invention is directed to a genetically modified bacterium capable of producing nicotinic acid riboside, wherein the bacterium comprises the following modifications: i) altered or increased L-aspartate oxidase activity, altered or increased quinolate synthase activity, altered or increased quinolate phosphoribosylpyrophosphate activity or combinations thereof, in a host with increased transcription of the gene encoding these activities or combinations thereof and ii) one or more additional modifications selected from the group consisting of: a) an altered nicotinic acid riboside kinase with a blocked or reduced activity; b) an altered nicotinic acid riboside phosphorylase with a blocked or reduced activity; c) an altered nicotinic acid riboside transport protein with a blocked or reduced activity; d) an altered nicotinate phosphoribosyl transferase with a blocked or reduced activity; e) an altered nicotinamide mononucleotide amidohydrolase with an added or increased activity; and f) an altered nicotinic acid mononucleotide hydrolase with an added or increased activity; wherein the bacterium with said at least one modification produces an increased amount of NaR than the bacterium without any of said modifications.

In another embodiment, the present invention is directed to a genetically modified bacterium capable of producing nicotinic acid riboside, wherein the bacterium comprises the following modifications: i) an altered nicotinic acid mononucleotide adenyltransferase with blocked or reduced activity; and ii) one or more additional modifications selected from the group consisting of: a) an altered nicotinic acid riboside kinase with a blocked or reduced activity; b) an altered nicotinic acid riboside phosphorylase with a blocked or reduced activity; c) an altered nicotinic acid riboside transport protein with a blocked or reduced activity; d) an altered nicotinic acid phosphoribosyl transferase with a blocked or reduced activity; e) an altered nicotinamide mononucleotide amidohydrolase with an added or increased activity; and f) an altered nicotinic acid mononucleotide hydrolase with an added or increased activity; wherein the bacterium with said at least one modification produces an increased amount of NaR than the bacterium without any of said modifications.

In one embodiment, the nicotinic acid mononucleotide adenyltransferase with reduced activity is exogenous to the host bacterium, i.e., not present in the cell prior to modification, having been introduced using recombination methods such as are described herein.

In another embodiment, the other proteins described above are endogenous to the host bacterium, i.e., present in the cell prior to modification, although alternations are made to increase or decrease the expression levels of the proteins. Examples of endogenous proteins for which expression levels are altered in the present invention include, but are not limited to, nicotinic acid mononucleotide adenyltransferase, negative regulator of NAD+ biosynthesis, nicotinic acid riboside kinase, nicotinic acid riboside phosphorylase, nicotinic acid riboside transport protein, nicotinic acid phosphoribosyl transferase, nicotinamide mononucleotide amidohydrolase, and nicotinic acid mononucleotide hydrolase.

The host bacterial cell may be genetically modified by any manner known to be suitable for this purpose by the person skilled in the art. This includes the introduction of the genes of interest, such as the gene encoding the nicotinic acid mononucleotide adenylating protein with reduced activity into a plasmid or cosmid or other expression vector which are capable of reproducing within the host cell. Alternatively, the plasmid or cosmid DNA or part of the plasmid or cosmid DNA or a linear DNA sequence may integrate into the host genome, for example by homologous recombination or random integration. To carry out genetic modification, DNA can be introduced or transformed into cells by natural uptake or by well-known processes such as electroporation. Genetic modification can involve expression of a gene under control of an introduced promoter. The introduced DNA may encode a protein which could act as an enzyme or could regulate the expression of further genes.

Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor Labs Press. The reference Sambrook et al., ibid., is incorporated by reference herein in its entirety.

A suitable polynucleotide may be introduced into the cell by random integration, homologous recombination and/or may form part of an expression vector comprising a combination of genes. Such an expression vector forms another aspect of the invention.

Suitable vectors for construction of such an expression vector are well known in the art and may be arranged to comprise the polynucleotide operably linked to one or more expression control sequences, so as to be useful to express the required enzymes in a host cell, for example a bacterium as described above. For example, promoters including, but not limited to, T7 promoter, pLac promoter, nudC promoter, ushA promoter, can be used in conjunction with endogenous genes and/or heterologous genes for modification of expression patterns of the targeted gene. Similarly, exemplary terminator sequences include, but are not limited to, the use of XPR1, XPR2, CPC1 terminator sequences.

In some embodiments, the recombinant or genetically modified bacterial cell, as mentioned throughout this specification, may be any gram-positive bacteria or gram-negative bacteria including but not limited to the genera Bacillus, Corynebacterium, Escherichia, Acinetobacter, Lactobacillus, Mycobacterium, Pseudomonas, and Ralstonia. In certain embodiments, exemplary species of bacteria include, but are not limited to, Bacillus subtilis, Corynebacterium glutamicum, Escherichia coli, Acinetobacter baylyi, and Ralstonia eutropha.

The genetically modified bacteria of the present disclosure also encompass bacteria comprising variants of the polypeptides as defined herein. As used herein, a “variant” means a polypeptide in which the amino acid sequence differs from the base sequence from which it is derived in that a substitution, insertion, and/or deletion of one or more (several) amino acid residues at one or more (several) positions are made. A substitution means a replacement of an amino acid occupying a position with a different amino acid; a deletion means removal of an amino acid occupying a position; and an insertion means adding 1-3 amino acids adjacent to an amino acid occupying a position.

The variants are functional variants in that the variant sequence has similar or identical functional enzyme activity characteristics to the enzyme having the native amino acid sequence specified herein.

For example, a functional variant of SEQ ID NO:4 has similar or identical nicotinic acid mononucleotide adenyltransferase activity characteristics as SEQ ID NO:4. An example may be that the rate of conversion by a functional variant of SEQ ID NO:4, of nicotinic acid mononucleotide to nicotinic acid adenine dinucleotide, may be the same or similar, although said functional variant may also provide other benefits. For example, at least about 80%, 90%, 95%, 96%, 97%, 98%, 99% or 100% the rate will be achieved when using the enzyme that is a functional variant of SEQ ID NO:4.

A functional variant or fragment of any of the above SEQ ID NO amino acid sequences, therefore, is any amino acid sequence which remains within the same enzyme category (i.e., has the same EC number). Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties.

Non-conservative substitutions are where amino acids are replaced with amino acids of a different type.

By “conservative substitution” is meant the substitution of an amino acid by another amino acid of the same class, in which the classes are defined as follows:

Class Amino Acid Examples:

Nonpolar: A, V, L, I, P, M, F, W

Uncharged polar: G, S, T, C, Y, N, Q

Acidic: D, E

Basic: K, R, H.

The present invention is also directed to nicotinic acid riboside compounds obtained from any of the above mentioned genetically modified bacterium.

The present invention is also directed to a composition comprising the nicotinic acid riboside compounds obtained from the above-mentioned genetically modified bacterium.

It will be appreciated that, the nicotinic acid ribose compounds isolated from the genetically modified bacteria of this invention can be reformulated into a final product. In some other embodiments of the disclosure, nicotinic acid ribose compounds produced by manipulated host cells as described herein are incorporated into a final product (e.g., food or feed supplement, pharmaceutical, etc.) in the context of the host cell. For example, host cells may be lyophilized, freeze dried, frozen or otherwise inactivated, and then whole cells may be incorporated into or used as the final product. The host cell may also be processed prior to incorporation in the product to increase bioavailability (e.g., via lysis).

In some embodiments of the disclosure, the produced nicotinic acid ribose compounds are incorporated into a component of food or feed (e.g., a food supplement). Types of food products into which nicotinic acid ribose compounds can be incorporated according to the present disclosure are not particularly limited, and include beverages such as milk, water, soft drinks, energy drinks, teas, and juices; confections such as jellies and biscuits; fat-containing foods and beverages such as dairy products; processed food products such as rice, bread, breakfast cereals, or the like. In some embodiments, the produced nicotinic acid ribose compounds is incorporated into a dietary supplement, such as, for example, a multivitamin.

### FIGURES

FIG. 1. Biochemical pathways for synthesizing quinolate from aspartate and dihydroxyacetone phosphate in the presence of NadA and NadB enzymes using E. coli nomenclature.

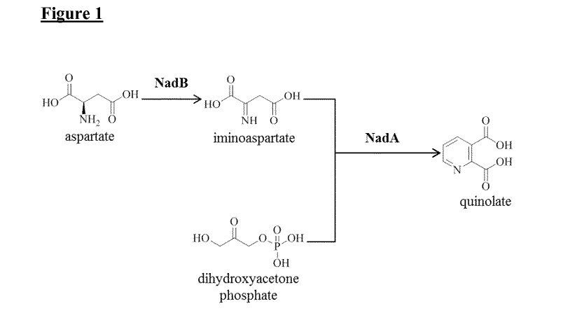

FIG. 2. Biochemical pathways and enzymes for synthesizing nicotinamide adenine dinucleotide using E. coli nomenclature.

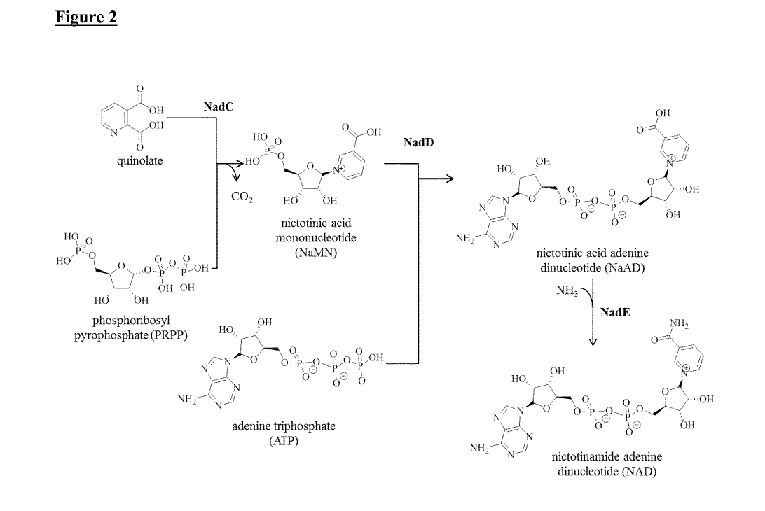

FIG. 3. Biochemical pathways useful for the production of nicotinic acid riboside from intermediates of NAD+ biosynthesis using E. coli nomenclature.

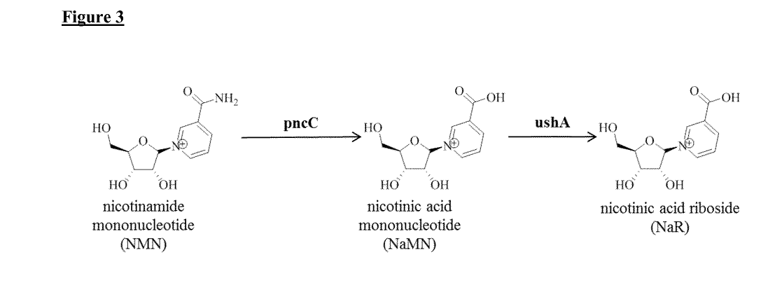

FIG. 4. biochemical pathways with undesirable activities for nicotinic acid riboside production using E. coli nomenclature. ATP: adenosine triphosphate; pRpp: 5-phospho-alpha-D-ribose 1-diphosphate; PPi: diphosphate; Pi: phosphate.

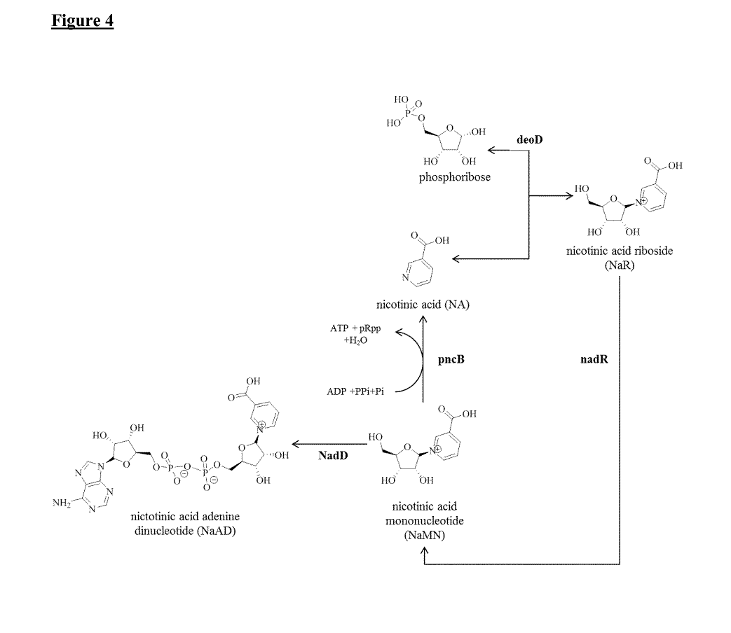

FIG. 5. Nicotinic acid riboside levels during fed-batch fermentation of strain ME517.

### OVERVIEW OF THE SEQUENCE LISTING

The nucleic acid sequences listed in the accompanying sequence listing are shown using standard letter abbreviation for nucleotide bases. Only one strand of each nucleic acid sequence is shown, but the complementary strand is understood to be included by any reference to the displayed strand. In the accompanying sequence listing:

SEQ ID NO: 1 is the amino acid sequence encoding the trifunctional Escherichia coli NadR enzyme (NMN synthetase, NaR kinase, negative regulator of NAD+ biosynthesis), which is a repressor protein.

SEQ ID NO: 2 is the amino acid sequence encoding the Bacillus subtilis YxrA enzyme, which is a repressor protein.

SEQ ID NO: 3 is the amino acid sequence encoding the Corynebacterium glutamicum CgR_1153 enzyme, which is a repressor protein.

SEQ ID NO: 4 is the amino acid sequence encoding the Escherichia coli NadD enzyme, which is a nicotinic acid mononucleotide adenyltransferase.

SEQ ID NO: 5 is the amino acid sequence encoding the Bacillus subtilis NadD enzyme, which is a nicotinic acid mononucleotide adenyltransferase.

SEQ ID NO: 6 is the amino acid sequence encoding the Corynebacterium glutamicum NadD Cg2584 enzyme, which is a nicotinic acid mononucleotide adenyltransferase.

SEQ ID NO: 7 is the amino acid sequence encoding the Escherichia coli DeoD enzyme, which is a nicotinic acid riboside phosphorylase.

SEQ ID NO:8 is the amino acid sequence encoding the Bacillus subtilis DeoD enzyme, which is a nicotinic acid riboside phosphorylase.

SEQ ID NO: 9 is the amino acid sequence encoding the Acinetobacter baylyi PnuC enzyme, which is a NaR transporter protein.

SEQ ID NO: 10 is the amino acid sequence encoding the Corynebacterium glutamicum PnuC enzyme, which is a NaR transporter protein.

SEQ ID NO: 11 is the amino acid sequence encoding the Escherichia coli PnuC enzyme, which is a NaR transporter protein.

SEQ ID NO: 12 is the amino acid sequence encoding the Escherichia coli UshA enzyme, which is a nicotinic acid mononucleotide hydrolase.

SEQ ID NO: 13 is the amino acid sequence encoding the Bacillus subtilis YfkN enzyme, which is a nicotinic acid mononucleotide hydrolase.

SEQ ID NO: 14 is the amino acid sequence encoding the Corynebacterium glutamicum Cg0397 enzyme, which is a nicotinic acid mononucleotide hydrolase.

SEQ ID NO: 15 is the amino acid sequence encoding the Escherichia coli PncB enzyme, which is a nicotinic acid phosphoribosyl transferase.

SEQ ID NO: 16 is the amino acid sequence encoding the Bacillus subtilis YueK enzyme, which is a nicotinic acid phosphoribosyl transferase.

SEQ ID NO: 17 is the amino acid sequence encoding the Corynebacterium glutamicum cg2774 enzyme, which is a nicotinic acid phosphoribosyl transferase.

SEQ ID NO:18 is the amino acid sequence encoding the Bacillus subtilis PupG enzyme, which is a nicotinic acid riboside phosphorylase.

SEQ ID NO:19 is the amino acid sequence encoding the Bacillus subtilis Pdp enzyme, which is a nicotinic acid riboside phosphorylase.

SEQ ID NO:20 is the amino acid sequence encoding the Escherichia coli PncC enzyme, which is a nicotinamide mononucleotide amidohydrolase.

SEQ ID NO:21 is the amino acid sequence encoding the Bacillus subtilis CinA enzyme, which is a nicotinamide mononucleotide amidohydrolase.

SEQ ID NO:22 is the amino acid sequence encoding the Corynebacterium glutamicum cg2153 enzyme, which is a nicotinamide mononucleotide amidohydrolase.

SEQ ID NO:23 is the amino acid sequence encoding the Escherichia coli NadA enzyme, which is a quinolate synthase.

SEQ ID NO:24 is the amino acid sequence encoding the Bacillus subtilis NadA enzyme, which is a quinolate synthase.

SEQ ID NO:25 is the amino acid sequence encoding the Corynebacterium glutamicum NadA enzyme, which is a quinolate synthase.

SEQ ID NO:26 is the amino acid sequence encoding the Escherichia coli NadB enzyme, which is a L-aspartate oxidase.

SEQ ID NO:27 is the amino acid sequence encoding the Bacillus subtilis NadB enzyme, which is a L-aspartate oxidase.

SEQ ID NO:28 is the amino acid sequence encoding the Escherichia coli NadC enzyme, which is a quinolate phosphoribosyl transferase.

SEQ ID NO:29 is the amino acid sequence encoding the Bacillus subtilis NadC enzyme, which is a quinolate phosphoribosyl transferase.

SEQ ID NO:30 is the amino acid sequence encoding the Corynebacterium glutamicum NadC enzyme, which is a quinolate phosphoribosyl transferase.

The following examples are intended to illustrate the invention without limiting its scope in any way.

### EXAMPLES

### Example 1: Disruption of the Negative Regulator of NAD+ Biosynthesis

All basic molecular biology and DNA manipulation procedures described herein are generally performed according to Sambrook et al. or Ausubel et al. (J. Sambrook, E. F. Fritsch, T. Maniatis (eds). 1989. Molecular Cloning: A Laboratory Manual. Cold Spring Harbor Laboratory Press: New York; and F. M. Ausubel, R. Brent, R. E. Kingston, D. D. Moore, J. G. Seidman, J. A. Smith, K. Struhl (eds.). 1998. Current Protocols in Molecular Biology. Wiley: New York).

Deletion of the gene encoding the negative regulator of NAD+ biosynthesis (nadR) is accomplished by lambda red mediated recombineering. An antibiotic gene, for example kanamycin resistance or chloramphenicol resistance, is PCR amplified using oligonucleotides that have flanks of 20-50 bps that are is homologous to the region upstream and downstream of the native nadR open reading frame. Alternatively, these flanks may be within the open reading frame, resulting in translation of a non-functional protein. The host strain, for example BL21(DE3), is prepared for lambda red recombineering by transformation with and induction of pKD46 as described (Datsenko and Wanner, 2000). Prepared cells are transformed by electroporation or chemical transformation and transformants are screened by PCR for successful disruption of the targeted gene.

### Example 2: Enhancement of Conversion from L-aspartic acid, dihydroxyacetone phosphate and 1-α-D-ribosylpyrophosphate to nicotinic acid mononucleotide

Aspartic acid is oxidized to iminosuccinic acid by the L-aspartate oxidase encoded in E. coli by the nadB gene. This example describes the construction of E. coli strains with alterations to the expression of the native nadB gene. The nadB gene is placed under the control of a strong constitutive promoter. DNA fragments encoding E. coli nadB gene are obtained either by PCR cloning or de novo DNA synthesis. In the case that DNA is obtained by synthesis, codon usage is optimized for expression in E. coli. DNA synthesis and optimization is carried out by GenScript, Inc. The nadB gene is expressed in E. coli under control of an inducible promoter. For example, the open reading frame is cloned into XhoI/NdeI-digested pET24, resulting in plasmid pET24-nadB. Transformation into a strain harboring the T7 polymerase, such as BL21(DE3), allows for IPTG induction of the nadB gene in order to promote NaR synthesis.

Alternatively, expression of the native nadB gene can also be altered by placing the nadB gene under the control of an inducible promoter. DNA fragments encoding inducible or constitutive promoters, such as the arabinose inducible pBAD or the constitutive pLac promoters, are obtained either by PCR cloning or de novo DNA synthesis. The promoter is fused downstream of an antibiotic gene, for example kanamycin resistance or chloramphenicol resistance, by fusion PCR. This marker-promoter cassette will contain flanks of 20-50 bps that are homologous to the region upstream of the native nadB promoter and to the first nucleotides of the nadB open reading frame. The host strain, for example BL21(DE3) is prepared for lambda red recombineering by transformation with and induction of pKD46 as described (Datsenko and Wanner, 2000). Prepared cells are transformed by electroporation or chemical transformation and transformants are screened by PCR for successful incorporation of the altered promoter sequence.

Quinolate synthase, which contains an iron-sulfur cluster, subsequently carries out the condensation and cyclization of iminosuccinic acid with dihydroxyacetone phosphate yielding quinolate and is encoded in E. coli by the nadA gene. This example describes the construction of E. coli strains with alterations to the expression of the native nadA gene.

The nadA gene is placed under the control of a strong constitutive promoter. DNA fragments encoding E. coli nadA gene are obtained either by PCR cloning or de novo DNA synthesis. In the case that DNA is obtained by synthesis, codon usage is optimized for expression in E. coli. DNA synthesis and optimization is carried out by GenScript, Inc. The nadA gene is expressed in E. coli under control of an inducible promoter. For example, the open reading frame is cloned into XhoI/NdeI-digested pET24, resulting in plasmid pET24-nadA. Transformation into a strain harboring the T7 polymerase, such as BL21(DE3), allows for IPTG induction of the nadA gene in order to promote NaR synthesis.

Alternatively, expression of the native nadA gene can also be altered by placing the nadB gene under the control of an inducible promoter. DNA fragments encoding inducible or constitutive promoters, such as the arabinose inducible pBAD or the constitutive pLac promoters, are obtained either by PCR cloning or de novo DNA synthesis. The promoter is fused downstream of an antibiotic gene, for example kanamycin resistance or chloramphenicol resistance, by fusion PCR. This marker-promoter cassette will contain flanks of 20-50 bps that are homologous to the region upstream of the native nadB promoter and to the first nucleotides of the nadA open reading frame. The host strain, for example BL21(DE3) is prepared for lambda red recombineering by transformation with and induction of pKD46 as described (Datsenko and Wanner, 2000, Proc. Natl. Acad. U.S.A 97(12):6640-5.). Prepared cells are transformed by electroporation or chemical transformation and transformants are screened by PCR for successful incorporation of the altered promoter sequence.

Quinolate phosphoribosyltransferase transfers the phosphoribosyl moiety from phosphoribosylpyrophosphate to the quinolate nitrogen and catalyzes the subsequent decarboxylation of the intermediate to produce nicotinic acid mononucleotide and is encoded in E. coli by the nadC gene. This example describes the construction of E. coli strains with alterations to the expression of the native nadC gene.

The nadC gene is placed under the control of a strong constitutive promoter. DNA fragments encoding E. coli nadC gene are obtained either by PCR cloning or de novo DNA synthesis. In the case that DNA is obtained by synthesis, codon usage is optimized for expression in E. coli. DNA synthesis and optimization is carried out by GenScript, Inc. The nadC gene is expressed in E. coli under control of an inducible promoter. For example, the open reading frame is cloned into XhoI/NdeI-digested pET24, resulting in plasmid pET24-nadC. Transformation into a strain harboring the T7 polymerase, such as BL21(DE3), allows for IPTG induction of the nadC gene in order to promote NAR synthesis.

Alternatively, expression of the native nadC gene can also be altered by placing the nadC gene under the control of an inducible promoter. DNA fragments encoding inducible or constitutive promoters, such as the arabinose inducible pBAD or the constitutive pLac promoters, are obtained either by PCR cloning or de novo DNA synthesis. The promoter is fused downstream of an antibiotic gene, for example kanamycin resistance or chloramphenicol resistance, by fusion PCR. This marker-promoter cassette will contain flanks of 20-50 bps that are homologous to the region upstream of the native nadC promoter and to the first nucleotides of the nadC open reading frame. The host strain, for example BL21(DE3) is prepared for lambda red recombineering by transformation with and induction of pKD46 as described (Datsenko and Wanner, 2000). Prepared cells are transformed by electroporation or chemical transformation and transformants are screened by PCR for successful incorporation of the altered promoter sequence.

Alternatively, expression nadA, nadB, and nadC is accomplished by expression in an operon. DNA fragments encoding E. coli nadA, nadB and nadC gene are obtained either by PCR cloning or de novo DNA synthesis. In the case that DNA is obtained by synthesis, codon usage is optimized for expression in E. coli. DNA synthesis and optimization is carried out by GenScript, Inc. Each gene is linked to a 5′ ribosome binding site and a 3′ terminator sequence. The operon is expressed in E. coli under control of an inducible promoter. For example, the open reading frame is cloned into XhoI/NdeI-digested pET24, resulting in plasmid pET24-nadABC. Transformation into a strain harboring the T7 polymerase, such as BL21(DE3), allows for IPTG induction of the nadABC gene in order to promote NAR synthesis.

### Example 3: Blockage or Reduction of Conversion from Nicotinic Acid Mononucleotide (NaMN) to Nicotinic Acid Adenine Dinucleotide (NaAD)

In E. coli and B. subtilis, NaMN is adenylated by the enzyme NadD. The enzymatic activity is essential for viability as all salvage and de novo pathways to NAD+ require this adenylation activity, however, accumulation of high levels of NaMN is desirable for NaR production. Replacement of the nadD gene with an inducible promoter would prevent these competing reactions, facilitating NaMN overproduction. Alternatively, alleles of the nadD gene with reduced enzyme activity have been characterized. Replacement of the native nadD gene with an allele with lower substrate affinity will decrease the effect of NadD enzyme activity on NaMN levels.

Many inducible promoters have been described in E. coli and are well characterized In this example, the IPTG inducible Lad promoter from E. coli is fused downstream of an antibiotic gene, for example kanamycin resistance or chloramphenicol resistance, by fusion PCR. This marker-pLac cassette will contain flanks of 20-50 bps that are homologous to the region surrounding the native nadD promoter. The host strain, for example BL21(DE3), is prepared for lambda red recombineering by transformation with induction of pKD46 as described (Datsenko and Wanner, 2000). Prepared cells are transformed by electroporation or chemical transformation and transformants are screened by PCR and sequencing for successful incorporation of the Lac promoter sequence in place of the native nadD promoter.

Alleles of E. coli nadD gene with lower activity but that are still able to support growth on minimal medium have been described, for example N40A or T11A. These mutations serve to increase the Km of NadD for NaMN, thereby increasing intracellular NaMN concentrations. Point mutations are introduced in vitro to the nadD gene via the Stragene QuickChange® site mutagenesis kit. The mutated gene is fused downstream of an antibiotic gene, for example kanamycin resistance or chloramphenicol resistance, by fusion PCR. This marker-nadD* cassette will contain flanks of 20-50 bps that are homologous to the region surrounding the native nadD gene. The host strain, for example BL21(DE3), is prepared for lambda red recombineering by transformation with induction of pKD46 as described (Datsenko and Wanner, 2000). Prepared cells are transformed by electroporation or chemical transformation and transformants are screened by PCR and sequencing for successful incorporation of the altered nadD sequence.

### Example 4: Enhancement of Conversion from Nicotinic Acid Mononucleotide (NaMN) to Nicotinic Acid Riboside (NaR)

Secreted NaMN is dephosphorylated to NaR via the periplasmic acid phosphatase encoded in E. coli by the ushA gene. This example describes the construction of E. coli strains with alterations to the expression of the native ushA gene.

To ensure NMN is dephosphorylated, the ushA gene is placed under the control of a strong constitutive promoter. DNA fragments encoding E. coli ushA gene are obtained either by PCR cloning or de novo DNA synthesis. In the case that DNA is obtained by synthesis, codon usage is optimized for expression in E. coli. DNA synthesis and optimization is carried out by GenScript, Inc. The ushA gene is expressed in E. coli under control of an inducible promoter. For example, the open reading frame is cloned into XhoI/NdeI-digested pET24, resulting in plasmid pET24-ushA. Transformation into a strain harboring the T7 polymerase, such as BL21 (DE3), allows for IPTG induction of the ushA gene in order to promote NAR synthesis.

Alternatively, expression of the native ushA gene can also be altered by placing the ushA gene under the control of an inducible promoter. DNA fragments encoding inducible or constitutive promoters, such as the arabinose inducible pBAD or the constitutive pLac promoters, are obtained either by PCR cloning or de novo DNA synthesis. The promoter is fused downstream of an antibiotic gene, for example kanamycin resistance or chloramphenicol resistance, by fusion PCR. This marker-promoter cassette will contain flanks of 20-50 bps that are homologous to the region upstream of the native ushA promoter and to the first nucleotides of the ushA open reading frame. The host strain, for example BL21(DE3) is prepared for lambda red recombineering by transformation with and induction of pKD46 as described (Datsenko and Wanner, 2000). Prepared cells are transformed by electroporation or chemical transformation and transformants are screened by PCR for successful incorporation of the altered promoter sequence.

### Example 5: Disruption of the Nicotinamide Adenine Dinucleotide (NAD) Salvage Pathway

Deletion of the gene encoding the nucleoside phosphorylase (deoD), the nicotinic acid/nicotinamide kinase (nadR) and the gene encoding the nicotinamide riboside uptake transporter (pnuC), either singly or in combination, is accomplished by lambda red mediated recombineering. An antibiotic gene, for example kanamycin resistance or chloramphenicol resistance, is PCR amplified using oligonucleotides that have flanks of 20-50 bps that are homologous to the region upstream and downstream of the native deoD, nadR, or pnuC open reading frame. Alternatively, these flanks may be within the open reading frame, resulting in translation of a non-functional protein. The host strain, for example BL21 (DE3), is prepared for lambda red recombineering by transformation with and induction of pKD46 as described (Datsenko and Wanner, 2000). Prepared cells are transformed by electroporation or chemical transformation and transformants are screened by PCR for successful disruption of the targeted gene. These knockouts may be combined with knockout of nadR (as disclosed in Example 1) or alterations to the NadD activity (as disclosed in Example 2) by assembly of the antibiotic gene with expression cassettes for these genes as described above for the promoter swap.

### Example 6: Cell Growth Condition and Protocols

E. coli strains engineered for the production of NaR are inoculated in LB medium with appropriate antibiotics and grown overnight at 37° C. Washed cells are resuspended in M9 medium with 5% glucose and grown for 3 days at 37° C. Where appropriate, IPTG is added to a concentration of 10-50 uM for induction.

### Example 7: Construction of a B. subtilis Strain with Increased Levels of NaR Production

Cassettes for the precise deletion of nadR, deoD, and pupG were constructed by long flanking PCR (LF-PCR). Flanking regions for each gene were obtained by amplification of BS168 genomic DNA (Roche High Pure PCR template preparation kit) with primers in Table 5, which were designed such that sequences homologous to the 5′ or 3′ region of the appropriate antibiotic resistance gene (spectinomycin, tetracycline, and neomycin, respectively, SEQ ID NOs: 48 to 50) were incorporated into the PCR product (Phusion Hot Start Flex DNA Polymerase, 200 nM each primer, initial denaturation 2 min @ 95 C, 30 cycles of: 30 sec @ 95 C; 20 sec @ 50 C; 60 sec @ 72 C, final hold 7 min at 72 C). Antibiotic resistance genes were similarly amplified with primers to incorporate sequences homologous to the 5′ and 3′ flanking regions. PCR products were gel purified and used for LF-PCR with appropriate primers (Table 5) (Phusion Hot Start Flex DNA Polymerase, 200 nM each primer, 150 ng each PCR product, initial denaturation 30 sec @ 98 C, 35 cycles of: 30 sec @ 98 C; 30 sec @ 55 C; 360 sec @ 72 C). LF-PCR product was purified and used for transformation of B. subtilis strains.

BS168 was transformed with LF-PCR product via natural transformation (“Molecular Biological Methods for Bacillus”. 1990. Edited by C. R Harwood and S. M. Cutting. John Wiley and Sons) yielding BS6209 (nadR::spe), ME479 (deoD::tet), and ME492 (pupG::neo). Genomic DNA (prepared as above) from ME492 was used to transform BS6209, yielding ME496 (nadR::spe pupG::neo). Genomic DNA (prepared as above) from ME479 was used to transform ME496, yielding ME517 (nadR::spe pupG::neo deoD::tet).

### Example 8: Production of Nicotinic Acid Riboside

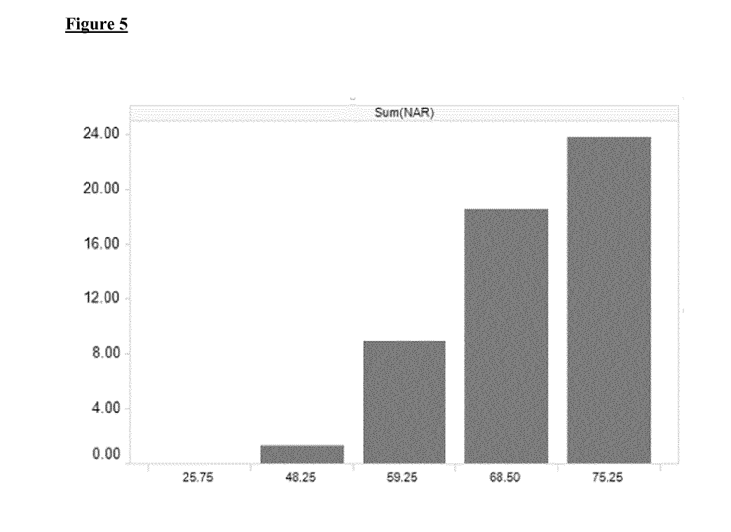

ME517 was grown for 8 hours at 37 C in a 500 mL baffled flask containing 50 mL of Seed (per liter: 30 g yeal infusion broth, 5 g bacto yeast extract, 10 g sorbitol, 1 drop Basildon 86/013 antifoam) medium. 1 mL of preculture was used to inoculate 300 mL of Seed medium in 2 L baffled flask and grown 16 hours at 37 C. 80 mL of this seed fermentation was used to inoculate production vessel (NBS) containing 1.2 L batch medium (1096 g H2O, 26.4 g dextrose, 9.6 g KH2PO4, 3.6 g MgSO4*7 H2O, 0.24 g CaCl₂)*H2O, 2.5 g L-tryptophan, 0.036 g MnCl2, 18 g MH4NO3, 0.12 g NaCitrate, 0.24 mL Clerol antifoam, 12 mg Na2EDTA*2 H2O, 57.5 mg ZnSO4*7 H2O, 3.2 mg MnSO4*H2O, 3.2 mg CuSO4, 4.8 mg Na2MoO4*2 H2O, CoCl2*6 H2O, 28 mg FeSO4*7 H2O). Agitation was initially set at 400 rpm, pH was maintained at 6.8 with addition of NH₄OH, and temperature was set at 37 C. During consumption of batch carbon, dO was maintained above 60% by increasing agitation as needed, and following consumption of batch carbon, dO was maintained at 60% by glucose feed. Nicotinic acid riboside was quantified as described below and results are shown in FIG. 5.

### Example 9: Detection of Nicotinic Acid Riboside in Production Cultures

Production cultures were diluted 10 fold in 20% acetonitrile, 0.1% formic acid in water and centrifuged.

NaR and intermediates were analyzed by liquid chromatography/mass spectrometry (LCMS). 20 μl of broth was diluted 1:50 in aqueous 5 mM ammonium acetate at pH 9.8 with 70% acetonitrile prior to centrifugation (5000×g, 10 m). The supernatant was removed and injected in 5 μl portions onto a HILIC HPLC column (Waters Atlantis C18, 2.1×150 mm). Compounds were eluted at a flow rate of 50 uL min-1, using a linear gradient from 5 mM ammonium acetate at pH 9.8 with 70% acetonitrile (A) to 5 mM ammonium acetate at pH 9.8 (B) over 20 minutes followed by a 5 minute hold in B and 10 minutes re-equilibration in A. Eluting compounds were detected with a triple quadropole mass spectrometer using positive electrospray ionization. The instrument is operated in MRM mode to detect NaR. NaR is quantified by comparison with standards (Sigma Aldrich) injected under the identical condition.

1-19. (canceled)

20. A genetically modified bacterium capable of producing nicotinic acid riboside (NaR), wherein the bacterium comprises at least one modification selected from a group consisting of:
a) blocking or reducing the activity of a protein which functions to repress NAD+ biosynthesis by repressing transcription of nadA, nadB, nadC genes or combinations thereof;
b) adding or increasing the transcription of a gene which encodes L-aspartate oxidase, quinolate synthase, quinolate phoshoribosyltransferase, or combinations thereof; and
c) blocking or reducing the activity of a protein which functions as a nicotinic acid mononucleotide adenyltransferase;
wherein the bacterium with said at least one modification produces an increased amount of NaR than the bacterium without any of said modifications; and
further comprising one or more additional modifications selected from the group consisting of:
d) blocking or reducing the activity of a protein which functions as a nicotinic acid riboside phosphorylase;
e) blocking or reducing the activity of a protein which functions as a nicotinic acid riboside kinase;
f) blocking or reducing the activity of a protein which functions as a nicotinic acid riboside transport protein;
g) blocking or reducing the activity of a protein which functions as a nicotinic acid phosphoribosyl transferase;
h) adding or increasing the activity of a protein which functions as a nicotinamide mononucleotide amidohydrolase; and
i) adding or increasing the activity of a protein which functions as a nicotinic acid mononucleotide hydrolase.

21. The bacterium of claim 20, wherein said protein which functions to repress NAD+ biosynthesis is a polypeptide comprising an amino acid sequence of any one of SEQ ID NO: 1, 2 or 3 or a variant thereof, wherein said polypeptide has an activity for repressing NAD+ biosynthesis.

22. The bacterium of claim 20, wherein said L-aspartate oxidase is a polypeptide comprising an amino acid sequence of any one of SEQ ID NO: 26 or 27 or a variant thereof, wherein said polypeptide has an activity for converting aspartic acid to iminosuccinic acid in an FAD dependent reaction.

23. The bacterium of claim 20, wherein said quinolate synthase is a polypeptide comprising an amino acid sequence of any one of SEQ ID NO: 23, 24 or 25 or a variant thereof, wherein said polypeptide has an activity for converting iminosuccinic acid and dihydroxyacetone phosphate to quinolate and phosphate.

24. The bacterium of claim 20, wherein said quinolate phosphoribosyltransferase is a polypeptide comprising an amino acid sequence of any one of SEQ ID NO: 28, 29 or 30 or a variant of said polypeptide, wherein said polypeptide has an activity for converting quinolate and phosphoribosylpyrophosphate to nicotinic acid mononucleotide and carbon dioxide.

25. The bacterium of claim 20, wherein the nicotinic acid mononucleotide adenyltransferase protein is a polypeptide comprising an amino acid sequence of any one of SEQ ID NO: 4, 5, or 6 or a variant of said polypeptide, wherein said polypeptide has a nicotinic acid mononucleotide adenyltransferase activity for converting nicotinic acid mononucleotide to nicotinic acid adenine dinucleotide.

26. The bacterium of claim 20, wherein the nicotinic acid riboside phosphorylase is a polypeptide comprising an amino acid sequence of any one of SEQ ID NOs: 7, 8, 18, or 19 or a variant of said polypeptide, wherein said polypeptide has a nucleoside cleavage activity for converting nicotinic acid riboside to nicotinic acid and ribose phosphate.

27. The bacterium of claim 20, wherein said nicotinic acid riboside kinase is a polypeptide comprising an amino acid sequence of SEQ ID NO: 1 or a variant of said polypeptide, wherein said polypeptide has an activity for converting nicotinic acid riboside to nicotinic acid mononucleotide.

28. The bacterium of claim 20, wherein the nicotinic acid riboside transporter is a polypeptide comprising an amino acid sequence of any one of SEQ ID NOs: 9, 10, or 11 or a variant of said polypeptide, wherein said polypeptide has a nicotinic acid riboside transport activity for importing nicotinic acid riboside.

29. The bacterium of claim 20, wherein the nicotinic acid phosphoribosyl transferase is a polypeptide comprising an amino acid sequence of any one of SEQ ID NOs: 15, 16, or 17 or a variant of said polypeptide, wherein said polypeptide has a nicotinic acid phosphoribosyl transferase activity for converting nicotinic acid, 5-phospho-ribose 1-diphosphate, and adenosine triphosphate to nicotinic acid mononucleotide, adenosine diphosphate, diphosphate and phosphate.

30. The bacterium of claim 20, wherein the nicotinamide mononucleotide amidohydrolase is a polypeptide comprising an amino acid sequence of any one of SEQ ID NOs: 20, 21, or 22 or a variant of said polypeptide, wherein said polypeptide has a nicotinamide mononucleotide amidohydrolase activity for converting nicotinamide mononucleotide to nicotinic acid mononucleotide.

31. The bacterium of claim 20, wherein the nicotinic acid mononucleotide hydrolase is a polypeptide comprising an amino acid sequence of any one of SEQ ID NOs: 12, 13, or 14 or a variant of said polypeptide, wherein said polypeptide has a nicotinic acid mononucleotide hydrolase activity for converting nicotinic acid mononucleotide to nicotinic acid riboside.

32. The bacterium of claim 1, wherein said bacterium is selected from a group consisting of: E. coli, B. subtilis, C. glutamicum, A. baylyi and R. eutropha.

33. A method for producing NaR, comprising: culturing a bacterium cell under conditions effective to produce NaR and recovering NaR from the medium and thereby producing NaR, wherein the host microorganism comprises at least one modification selected from the group consisting of:
a) blocking or reducing the activity of a protein which functions to repress NAD+ biosynthesis by repressing transcription of nadA, nadB, nadC genes or combinations thereof;
b) adding or increasing the transcription of a gene which encodes L-aspartate oxidase, quinolate synthase, quinolate phosphoribosyltransferase, or combinations thereof; and
c) blocking or reducing the activity of a protein which functions as a nicotinic acid mononucleotide adenyltransferase.

34. The method of claim 33, wherein the bacterium cell further comprises at least one modification selected from the group consisting of:
d) blocking or reducing the activity of a protein which functions as a nicotinate riboside phosphorylase;
e) blocking or reducing the activity of a protein which functions as a nicotinic acid riboside kinase;
f) blocking or reducing the activity of a protein which functions as a nicotinic acid riboside transport protein;
g) blocking or reducing the activity of a protein which functions as a nicotinic acid phosphoribosyl transferase;
h) adding or increasing the activity of a protein which functions as a nicotinamide mononucleotide amidohydrolase; and
i) adding or increasing the activity of a protein which functions as a nicotinic acid mononucleotide hydrolase.


SEQUENCE LISTING (30 sequences)

SEQ ID NO: 1 - protein, 410 aa, Escherichia coli
MSSFDYLKTAIKQQGCTLQQVADASGMTKGYLSQLLNAKIKSPSAQKLEALHRFLGLEFPRQKKTIGVVFGKFYPLHTGHIYLIQRACSQVDELHIIMGFDDTRDRALFEDSAMSQQPTVPDRLRWLLQTFKYQKNIRIHAFNEEGMEPYPHGWDVWSNGIKKFMAEKGIQPDLIYTSEEADAPQYMEHLGIETVLVDPKRTFMSISGAQIRENPFRYWEYIPTEVKPFFVRTVAILGGESSGKSTLVNKLANIFNTTSAWEYGRDYVFSHLGGDEIALQYSDYDKIALGHAQYIDFAVKYANKVAFIDTDFVTTQAFCKKYEGREHPFVQALIDEYRFDLVILLENNTPWVADGLRSLGSSVDRKEFQNLLVEMLEENNIEFVRVEEEDYDSRFLRCVELVREMMGEQR

SEQ ID NO: 2 - protein, 180 aa, Bacillus subtilis
MTEELKLMGANRRDQLLLWLKESKSPLTGGELAKKANVSRQVIVQDISLLKAKNVPIIATSQGYVYMDAAAQQHQQAERIIACLHGPERTEEELQLIVDEGVTVKDVKIEHPVYGDLTAAIQVGTRKEVSHFIKKINSTNAAYLSQLTDGVHLHTLTAPDEHRIDQACQALEEAGILIKD

SEQ ID NO: 3 - protein, 214 aa, Corynebacterium glutamicum
MPASPEIQMAVSTIIFALRPGPQDLPSLWAPFVPRTREPHLNKWALPGGWLPPHEELEDAAARTLAETTGLHPSYLEQLYTFGKVDRSPTGRVISVVYWALVRADEALKAIPGENVQWFPADHLPELAFDHNDIVKYALERLRTKVEYSEIAHSFLGETFTIAQLRSVHEAVLGHKLDAANFRRSVATSPDLIDTGEVLAGTPHRPPKLFRFQR

SEQ ID NO: 4 - protein, 213 aa, Escherichia coli
MKSLQALFGGTFDPVHYGHLKPVETLANLIGLTRVTIIPNNVPPHRPQPEANSVQRKHMLELAIADKPLFTLDERELKRNAPSYTAQTLKEWRQEQGPDVPLAFIIGQDSLLTFPTWYEYETILDNAHLIVCRRPGYPLEMAQPQYQQWLEDHLTHNPEDLHLQPAGKIYLAETPWFNISATIIRERLQNGESCEDLLPEPVLTYINQQGLYR

SEQ ID NO: 5 - protein, 189 aa, Bacillus subtilis
MKKIGIFGGTFDPPHNGHLLMANEVLYQAGLDEIWFMPNQIPPHKQNEDYTDSFHRVEMLKLAIQSNPSFKLELVEMEREGPSYTFDTVSLLKQRYPNDQLFFIIGADMIEYLPKWYKLDELLNLIQFIGVKRPGFHVETPYPLLFADVPEFEVSSTMIRERFKSKKPTDYLIPDKVKKYVEENGLYES

SEQ ID NO: 6 - protein, 226 aa, Corynebacterium glutamicum
MRTLYCPLMTTTVKRRARIGIMGGTFDPIHNGHLVAGSEVADRFDLDLVVYVPTGQPWQKANKKVSPAEDRYLMTVIATASNPRFMVSRVDIDRGGDTYTIDTLQDLSKQYPDAQLYFITGADALAQIVTWRDWEKTFELAHFVGVTRPGYELDGNIIPEMHQDRVSLVDIPAMAISSTDCRERSSEERPVWYLVPDGVVQYIAKRQLYRPEGSDKDMDPKGQNQA

SEQ ID NO: 7 - protein, 239 aa, Escherichia coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 8 - protein, 233 aa, Bacillus subtilis
MSVHIGAEKGQIADTVLLPGDPLRAKFIAETYLENVECYNEVRGMYGFTGTYKGKKISVQGTGMGVPSISIYVNELIQSYDVQNLIRVGSCGAIRKDVKVRDVILAMTSSTDSQMNRVAFGSVDFAPCADFELLKNAYDAAKDKGVPVTVGSVFTADQFYNDDSQIEKLAKYGVLGVEMETTALYTLAAKHGRKALSILTVSDHVLTGEETTAEERQTTFHDMIEVALHSVSQ

SEQ ID NO: 9 - protein, 191 aa, Acinetobacter baylyi
MSPLEIFAVIISVIGVALTIKRNMWCWGFNFLAFILYGYLFFSFKLYGETILQGFFIIINFYGFYYWLKGKQTEHEIRIVAIPAKTVIIQMLLAALGGLIFGLSLKHFTDAAVPMLDSQLAAFSLLATYWTSRKHIATWVLWVFVDIVYVGMFIYKDLYLTAGLYAAFVVMAAFGWWQWEQVKRKQRSGLI

SEQ ID NO: 10 - protein, 230 aa, Corynebacterium glutamicum
MNPITELLDATLWIGGVPILWREIIGNVFGLFSAWAGMRRIVWAWPIGIIGNALLFTVFMGGLFHTPQNLDLYGQAGRQIMFIIVSGYGWYQWSAAKRRALTPENAVAVVPRWASTKERAGIVIAAVVGTLSFAWIFQALGSWGPWADAWIFVGSILATYGMARGWTEFWLIWIAVDIVGVPLLLTAGYYPSAVLYLVYGAFVSWGFVVWLRVQKADKARALEAQESVTV

SEQ ID NO: 11 - protein, 239 aa, Escherichia coli
MDFFSVQNILVHIPIGAGGYDLSWIEAVGTIAGLLCIGLASLEKISNYFFGLINVTLFGIIFFQIQLYASLLLQVFFFAANIYGWYAWSRQTSQNEAELKIRWLPLPKALSWLAVCVVSIGLMTVFINPVFAFLTRVAVMIMQALGLQVVMPELQPDAFPFWDSCMMVLSIVAMILMTRKYVENWLLWVIINVISVVIFALQGVYAMSLEYIILTFIALNGSRMWINSARERGSRALSH

SEQ ID NO: 12 - protein, 550 aa, Escherichia coli
MKLLQRGVALALLTTFTLASETALAYEQDKTYKITVLHTNDHHGHFWRNEYGEYGLAAQKTLVDGIRKEVAAEGGSVLLLSGGDINTGVPESDLQDAEPDFRGMNLVGYDAMAIGNHEFDNPLTVLRQQEKWAKFPLLSANIYQKSTGERLFKPWALFKRQDLKIAVIGLTTDDTAKIGNPEYFTDIEFRKPADEAKLVIQELQQTEKPDIIIAATHMGHYDNGEHGSNAPGDVEMARALPAGSLAMIVGGHSQDPVCMAAENKKQVDYVPGTPCKPDQQNGIWIVQAHEWGKYVGRADFEFRNGEMKMVNYQLIPVNLKKKVTWEDGKSERVLYTPEIAENQQMISLLSPFQNKGKAQLEVKIGETNGRLEGDRDKVRFVQTNMGRLILAAQMDRTGADFAVMSGGGIRDSIEAGDISYKNVLKVQPFGNVVVYADMTGKEVIDYLTAVAQMKPDSGAYPQFANVSFVAKDGKLNDLKIKGEPVDPAKTYRMATLNFNATGGDGYPRLDNKPGYVNTGFIDAEVLKAYIQKSSPLDVSVYEPKGEVSWQ

SEQ ID NO: 13 - protein, 1462 aa, Bacillus subtilis
MRIQKRRTHVENILRILLPPIMILSLILPTPPIHAEESAAPQVHLSILATTDIHANMMDYDYYSDKETADFGLARTAQLIQKHREQNPNTLLVDNGDLIQGNPLGEYAVKYQKDDIISGTKTHPIISVMNALKYDAGTLGNHEFNYGLDFLDGTIKGADFPIVNANVKTTSGENRYTPYVINEKTLIDENGNEQKVKVGYIGFVPPQIMTWDKKNLEGQVQVQDIVESANETIPKMKAEGADVIIALAHTGIEKQAQSSGAENAVFDLATKTKGIDAIISGHQHGLFPSAEYAGVAQFNVEKGTINGIPVVMPSSWGKYLGVIDLKLEKADGSWKVADSKGSIESIAGNVTSRNETVTNTIQQTHQNTLEYVRKPVGKTEADINSFFAQVKDDPSIQIVTDAQKWYAEKEMKDTEYKNLPILSAGAPFKAGGRNGANYYTNIPAGDLAIKNVGDLYLYDNTVQIVKLTGSEVKDWLEMSAGQFNQIDPAKGGDQALLNENFRSYNFDVIDGVTYQVDVTKPAKYNENGKVINADSSRIINLSYEGKPISPSQEFLVVTNNYRASGGGGFPHLTSDKIVHGSAVENRQVLMDYIIEQKTVNPKADNNWSIAPVSGTNLTFESSLLAKPFADKADDVAYVGKSANEGYGVYKLQFDDDSNPDPPKDGLWDLTVMHTNDTHAHLDDAARRMTKINEVRSETNHNILLDAGDVFSGDLYFTKWNGLADLKMMNMMGYDAMTFGNHEFDKGPTVLSDFLSGNSATVDPANRYHFEAPEFPIVSANVDVSNEPKLKSFVKKPQTFTAGEKKEAGIHPYILLDVDGEKVAVFGLTTEDTATTSSPGKSIVFNDAFETAQNTVKAIQEEEKVNKIIALTHIGHNRDLELAKKVKGIDLIIGGHTHTLVDKMEVVNNEEPTIVAQAKEYGQFLGRVDVAFDEKGVVQTDKSNLSVLPIDEHTEENPEAKQELDQFKNELEDVKNEKVGYTDVALDGQREHVRTKETNLGNFIADGMLAKAKEAAGARIAITNGGGIRAGIDKGDITLGEVLNVMPFGNTLYVADLTGKQIKEALEQGLSNVENGGGAFPQVAGIEYTFTLNNKPGHRVLEVKIESPNGDKVAINTDDTYRVATNNFVGAGGDGYSVFTEASHGEDLGYVDYEIFTEQLKKLGNKVSPKVEGRIKEVFLPTKQKDGSWTLDEDKFAIYAKNANTPFVYYGIHEGSQEKPINLKVKKDQVKLLKERESDPSLTMFNYWYSMKMPMANLKTADTAIGIKSTGELDVSLSDVYDFTVKQKGKEIKSFKEPVQLSLRMFDIEEAHNPAIYHVDRKKKAFTKTGHGSVDDDMVTGYTNHFSEYTILNSGSNNKPPAFPSDQPTGGDDGNHGGGSDKPGGKQPTDGNGGNDTPPGTQPTNGSGGNGSGGSGTDGPAGGLLPDTATSMYSILLAGFLISALGTAMYLHQRRKQNRANQA

SEQ ID NO: 14 - protein, 694 aa, Corynebacterium glutamicum
MKRLSRAALAVVATTAVSFSALAVPAFADEASNVELNILGVTDFHGHIEQKAVKDDKGVITGYSEMGASGVACYVDAERADNPNTRFITVGDNIGGSPFVSSILKDEPTLQALSAIGVDASALGNHEFDQGYSDLVNRVSLDGSGSAKFPYLGANVEGGTPAPAKSEIIEMDGVKIAYVGAVTEETATLVSPAGIEGITFTGDIDAINAEADRVIEAGEADVVIALIHAEAAPTDLFSNNVDVVFSGHTHFDYVAEGEARGDKQPLVVIQGHEYGKVISDVEISYDREAGKITNIEAKNVSATDVVENCETPNTAVDAIVAAAVEAAEEAGNEVVATIDNGFYRGADEEGTTGSNRGVESSLSNLIAEAGLWAVNDATILNADIGIMNAGGVRADLEAGEVTFADAYATQNFSNTYGVREVSGAQFKEALEQQWKETGDRPRLALGLSSNVQYSYDETREYGDRITHITFNGEPMDMKETYRVTGSSFLLAGGDSFTAFAEGGPIAETGMVDIDLFNNYIAAHPDAPIRANQSSVGIALSGPAVAEDGTLVPGEELTVDLSSLSYTGPEAKPTTVEVTVGTEKKTADVDNTIVPQFDSTGKATVTLTVPEGATSVKIATDNGTTFELPVTVNGEGNNDDDDDKEQQSSGSSDAGSLVAVLGVLGALGGLVAFFLNSAQGAPFLAQLQAMFAQFM

SEQ ID NO: 15 - protein, 400 aa, Escherichia coli
MTQFASPVLHSLLDTDAYKLHMQQAVFHHYYDVHVAAEFRCRGDDLLGIYADAIREQVQAMQHLRLQDDEYQWLSALPFFKADYLNWLREFRFNPEQVTVSNDNGKLDIRLSGPWREVILWEVPLLAVISEMVHRYRSPQADVAQALDTLESKLVDFSALTAGLDMSRFHLMDFGTRRRFSREVQETIVKRLQQESWFVGTSNYDLARRLSLTPMGTQAHEWFQAHQQISPDLANSQRAALAAWLEEYPDQLGIALTDCITMDAFLRDFGVEFASRYQGLRHDSGDPVEWGEKAIAHYEKLGIDPQSKTLVFSDNLDLRKAVELYRHFSSRVQLSFGIGTRLTCDIPQVKPLNIVIKLVECNGKPVAKLSDSPGKTICHDKAFVRALRKAFDLPHIKKAS

SEQ ID NO: 16 - protein, 490 aa, Bacillus subtilis
MLEYGFKDDSLSLHTDLYQINMAETYWRDGIHEKKAIFELFFRRLPFENGYAVFAGLEKAIEYLENFKFTDSDLSYLQDELGYHEDFIEYLRGLSFTGSLYSMKEGELVFNNEPIMRVEAPLVEAQLIETALLNIVNYQTLIATKAARIKGVIGDEVALEFGTRRAHEMDAAMWGARAALIGGFSATSNVRAGKRFNIPVSGTHAHALVQAYRDEYTAFKKYAETHKDCVFLVDTYDTLRSGMPNAIRVAKEFGDRINFIGIRLDSGDLAYLSKKARKMLDEAGFTDAKVIASSDLDEHTIMNLKAQGARIDVWGVGTKLITAYDQPALGAVYKLVAIEEDGKMVDTIKISSNPEKVTTPGRKKVYRIINQSNHHSEGDYIALYDEQVNDQKRLRMFHPVHTFISKFVTNFYAKDLHELIFEKGILCYQNPEISDIQQYVQDNLSLLWEEYKRISKPEEYPVDLSEDCWSNKMQRIHEVKSRIEEELEEE

SEQ ID NO: 17 - protein, 446 aa, Corynebacterium glutamicum
MNTNPSEFSSNRSTALLTDKYELTMLQAALADGSAERPSTFEVFSRRLPNERRYGVVAGTARVLKAIRDFVFTEEQLADLDFLDDRTLEYLRNYRFTGQVDGYREGEIYFPQSPLLTVRGTFAECVILETVILSIMNADSAVASAAARMVTAADGRPIIEMGSRRTHEYSAVTASRAAYLAGFSTTSNLEAAYRYGIPASGTSAHAWTLLHINDDGTPNEAAAFKAQVESLGVDTTLLVDTYDITQGVATAIEVAGPDLGGVRIDSGDLGVLARKVRKQLDDLNAHNTKIVVSSDLDEFAIAGLRGEPVDVFGVGTSVVTGSGAPTAGLVYKIVEVAGHPVAKRSRNKESYGGGKKAVRTHRKSGTAIEEIVYPFNAEAPDTGKLDTLSLTIPLMRDGEIVPGLPTLEDSRAYLAKQLVSLPWEGLALSRDEPVLHTRFVGFPPAA

SEQ ID NO: 18 - protein, 271 aa, Bacillus subtilis
MKDRIERAAAFIKQNLPESPKIGLILGSGLGILADEIENPVKLKYEDIPEFPVSTVEGHAGQLVLGTLEGVSVIAMQGRFHFYEGYSMEKVTFPVRVMKALGVEALIVTNAAGGVNTEFRAGDLMIITDHINFMGTNPLIGPNEADFGARFPDMSSAYDKDLSSLAEKIAKDLNIPIQKGVYTAVTGPSYETPAEVRFLRTMGSDAVGMSTVPEVIVANHAGMRVLGISCISNAAAGILDQPLSHDEVMEVTEKVKAGFLKLVKAIVAQYE

SEQ ID NO: 19 - protein, 433 aa, Bacillus subtilis
MRMVDIIIKKQNGKELTTEEIQFFVNGYTDGSIPDYQASALAMAIFFQDMSDRERADLTMAMVNSGETIDLSAIEGIKVDKHSTGGVGDTTTLVLAPLVAALDVPVAKMSGRGLGHTGGTIDKLEAIMGFHVELTKDEFIKLVNRDKVAVIGQSGNLTPADKKLYALRDVTGTVNSIPLIASSIMSKKIAAGADAIVLDVKTGAGAFMKTEEDAAELAKAMVRIGNNVGRQTMAVISDMSQPLGFAIGNALEVKEAIDTLKGEGPEDLHELVLTLGSQMVVLAKKADTLDEARAKLEEVMKNGKALEKFKDFLKNQGGDSSIVDDPSKLPQAAYQIDVPAKEAGVVSEIVADEIGVAAMLLGAGRATKEDEIDLAVGIMLRKKVGDKVEKGEPLVTLYANRENVDEVIAKVYDNIRIAAEAKAPKLIHTLITE

SEQ ID NO: 20 - protein, 165 aa, Escherichia coli
MTDSELMQLSEQVGQALKARGATVTTAESCTGGWVAKVITDIAGSSAWFERGFVTYSNEAKAQMIGVREETLAQHGAVSEPVVVEMAIGALKAARADYAVSISGIAGPDGGSEEKPVGTVWFAFATARGEGITRRECFSGDRDAVRRQATAYALQTLWQQFLQNT

SEQ ID NO: 21 - protein, 416 aa, Bacillus subtilis
MEFPKKAEIIAVGSELLLGQIANTNAQFISKQLAEIGVHVFYHTAVGDNPERLKQVIRIAEERSDFIIFSGGLGPTKDDLTKETIANTLGRPLVLNDEAFQSIEDYPKRTKRTMSPNNRKQALVIEGSDVLANHFGMAPGMLTEHESRYYMLLPGPPSELRPMFENEAKPLLLKKMGSNEKIVSTVLRFFGIGESQLEPDLEDIIDAQTNPTIAPLAADGEVTLRLTAKHADEKETERLLKETEAVILERVGEFFYGYDDTSLVKELSIACKEKGITISAAESFTGGLFSEWLTDHSGASKLFAGGVVCYTNDVKQNVLGVKKETLDRFGAVSKECASELAKGVQKLTGSDIGISFTGVAGPDAQEGHEPGHVFIGISANGKEEVHEFHFAGSRTGIRKRGAKYGCHLILKLLEQK

SEQ ID NO: 22 - protein, 172 aa, Corynebacterium glutamicum
MSENLAGRVVELLKSRGETLAFCESLTAGLASATIAEIPGASVVLKGGLVTYATELKVALAGVPQELIDAHGVVSPQCARAMATGAAHRCQADWAVSLTGVAGPSKQDGHPVGEVWIGVAGPAHFGASGTIDAYRAFESEQQVILAELGRHHIRESAVQQSFRLLIDHIESQ

SEQ ID NO: 23 - protein, 347 aa, Escherichia coli
MSVMFDPDTAIYPFPPKPTPLSIDEKAYYREKIKRLLKERNAVMVAHYYTDPEIQQLAEETGGCISDSLEMARFGAKHPASTLLVAGVRFMGETAKILSPEKTILMPTLQAECSLDLGCPVEEFNAFCDAHPDRTVVVYANTSAAVKARADWVVTSSIAVELIDHLDSLGEKIIWAPDKHLGRYVQKQTGGDILCWQGACIVHDEFKTQALTRLQEEYPDAAILVHPESPQAIVDMADAVGSTSQLIAAAKTLPHQRLIVATDRGIFYKMQQAVPDKELLEAPTAGEGATCRSCAHCPWMAMNGLQAIAEALEQEGSNHEVHVDERLRERALVPLNRMLDFAATLRG

SEQ ID NO: 24 - protein, 368 aa, Bacillus subtilis
MSILDVIKQSNDMMPESYKELSRKDMETRVAAIKKKFGSRLFIPGHHYQKDEVIQFADQTGDSLQLAQVAEKNKEADYIVFCGVHFMAETADMLTSEQQTVVLPDMRAGCSMADMADMQQTNRAWKKLQHIFGDTIIPLTYVNSTAEIKAFVGKHGGATVTSSNAKKVLEWAFTQKKRILFLPDQHLGRNTAYDLGIALEDMAVWDPMKDELVAESGHTNVKVILWKGHCSVHEKFTTKNIHDMRERDPDIQIIVHPECSHEVVTLSDDNGSTKYIIDTINQAPAGSKWAIGTEMNLVQRIIHEHPDKQIESLNPDMCPCLTMNRIDLPHLLWSLEQIEKGEPSGVIKVPKAIQEDALLALNRMLSIT

SEQ ID NO: 25 - protein, 428 aa, Corynebacterium glutamicum
MTTSITPSVNLALKNANSCNSELKDGPWFLDQPGMPDVYGPGASQNDPIPAHAPRQQVLPEEYQRASDDELHRRIREAKDTLGDKVVILGHFYQRDEVIQHADFVGDSFQLARAAKTRPEAEAIVFCGVHFMAETADLLSTDEQSVILPNLAAGCSMADMADLDSVEDCWEQLTSIYGDDTLIPVTYMNSSAALKGFVGEHGGIVCTSSNARSVLEWAFERGQRVLFFPDQHLGRNTAKAMGIGIDQMPLWNPNKPLGGNTVSELENAKVLLWHGFCSVHKRFTVEQINKARAEYPDVHVIVHPESPMPVVDAADSSGSTDFIVKAIQAAPAGSTFAIGTEINLVQRLAAQYPQHTIFCLDPVICPCSTMYRIHPGYLAWALEELVAGNVINQISVSESVAAPARVALERMLSVVPAAPVTPSSSKDA

SEQ ID NO: 26 - protein, 540 aa, Escherichia coli
MNTLPEHSCDVLIIGSGAAGLSLALRLADQHQVIVLSKGPVTEGSTFYAQGGIAAVFDETDSIDSHVEDTLIAGAGICDRHAVEFVASNARSCVQWLIDQGVLFDTHIQPNGEESYHLTREGGHSHRRILHAADATGREVETTLVSKALNHPNIRVLERSNAVDLIVSDKIGLPGTRRVVGAWVWNRNKETVETCHAKAVVLATGGASKVYQYTTNPDISSGDGIAMAWRAGCRVANLEFNQFHPTALYHPQARNFLLTEALRGEGAYLKRPDGTRFMPDFDERGELAPRDIVARAIDHEMKRLGADCMFLDISHKPADFIRQHFPMIYEKLLGLGIDLTQEPVPIVPAAHYTCGGVMVDDHGRTDVEGLYAIGEVSYTGLHGANRMASNSLLECLVYGWSAAEDITRRMPYAHDISTLPPWDESRVENPDERVVIQHNWHELRLFMWDYVGIVRTTKRLERALRRITMLQQEIDEYYAHFRVSNNLLELRNLVQVAELIVRCAMMRKESRGLHFTLDYPELLTHSGPSILSPGNHYINR

SEQ ID NO: 27 - protein, 531 aa, Bacillus subtilis
MSKKTIAVIGSGAAALSLAAAFPPSYEVTVITKKSVKNSNSVYAQGGIAAAYAKDDSIEAHLEDTLYAGCGHNNLAIVADVLHDGKMMVQSLLERGFPFDRNERGGVCLGREGAHSYNRIFHAGGDATGRLLIDYLLKRINSKIKLIENETAADLLIEDGRCIGVMTKDSKGRLKVRHADEVVLAAGGCGNLFLHHTNDLTVTGDGLSLAYRAGAELTDLEFTQFHPTLLVKNGVSYGLVSEAVRGEGGCLVDENGRRIMAERHPLGDLAPRDIVSRVIHEEMAKGNRVYIDFSAISDFETRFPTITAICEKAGIDIHSGKIPVAPGMHFLMGGVSVNRWGETTVPGLYAIGETACSGLHGANRLASNSLLEALVFGKRAAEHIIQKPVYNRQYQSGLETSVFYEVPDIEGHELQSKMTSHMSILREQSSLIELSIWLHTLPFQEVNVKDITIRQMELSHLWQTAKLMTFSALLREESRGAHFRTDFPHAEVSWQGRQIVHTKKGTKIRKNEGIWNNESFTAEKITESLFS

SEQ ID NO: 28 - protein, 297 aa, Escherichia coli
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 29 - protein, 289 aa, Bacillus subtilis
MNHLQLKKLLNHFFLEDIGTGDLTSQSIFGEQSCEAEIVAKSEGIFAGAAIIKEGFSLLDENVQSILHKKDGDMLHKGEVIAELHGPAAALLSGERVVLNLIQRLSGIATMTREAVRCLDDEQIKICDTRKTTPGLRMLEKYAVRAGGGYNHRFGLYDGIMIKDNHIAACGSILEACKKARQAAGHMVNIEVEIETEEQLREAIAAGADVIMFDNCPPDTVRHFAKLTPANIKTEASGGITLESLPAFKGTGVNYISLGFLTHSVKSLDISMDVTLSNESVEECCYVNS

SEQ ID NO: 30 - protein, 279 aa, Corynebacterium glutamicum
MTTHIDRIVGAALSEDAPWGDITSDTFIPGSAQLSAKVVAREPGVFSGQALFDASFRLVDPRINASLKVADGDSFETGDILGTITGSARSILRSERIALNFIQRTSGIATLTSCYVAEVKGTKARIVDTRKTTPGLRIIERQAVRDGGGFNHRATLSDAVMVKDNHLAAIASQGLSITEALSNMKAKLPHTTHVEVEVDHIEQIEPVLAAGVDTIMLDNFTIDQLIEGVDLIGGRALVEASGGVNLNTAGKIASTGVDVISVGALTHSVHALDLGLDIF